(19) United States
(12) Patent Application Publication
(10) Pub. No.: US 2011/0039341 A1
(43) Pub. Date: Feb. 17, 2011

(54) Method for detecting nucleotide polymor-phisms using semiconductor particles

(76) Inventors: Jianqin Liu, Milwaukee, WI (US); Tijana Rajh, Naperville, IL (US); Zoran Saponjic, Belgrade (YU); Ligang Zhang, Westmont, IL (US)

Correspondence Address: MICHAEL J. CHERSKOV, 123 WEST MADISON STREET, SUITE 400, CHICAGO, IL, 60602, US

(21) Appl. No.: 11/517,181

(22) Filed: Sep. 7, 2006

Related U.S. Application Data
(63) Continuation-in-part of application No. 11/438,180, filed on May 22, 2006, now abandoned.
Application No. 11/438,180 is a continuation-in-part of application No. 10/823,509, filed on Apr. 12, 2004, now abandoned.
Application No. 10/823,509 is a continuation-in-part of application No. 10/755,045, filed on Jan. 9, 2004, now abandoned.
Application No. 10/755,045 is a continuation of application No. 09/606,429, filed on Jun. 28, 2000, now Pat. No. 6,677,606.

(51) Int. Cl. G01N 33/00

(52) U.S. Cl. 436/86; 436/94

(57) ABSTRACT

A method for detecting anomalies in genetic material is provided, the method comprising supplying the genetic material; establishing electronic communication between the genetic material and a semi-conductor particle so as to create a composite; contacting the composite with a first metal ion; and subjecting the contacted composite to energy in an amount and for a time sufficient to reduce the first metal ion to a first elemental metal. Also provided is a device for detecting single nucleotide mismatching in genetic material, the device comprising a semiconductor particle; a ligand attached to the particle; the genetic material in electronic communication with the ligand so as to form an organic-inorganic composite; metal ion in electronic communication with the composite, such that the metal ion reduces to elemental metal when the semiconductor particle is exposed to radiation of a predetermined energy level.

### PRIORITY CLAIM

This Utility Patent Application is a Continuation In Part of U.S. Utility patent application Ser. No. 11/438,180, filed on May 22, 2006, which is a Continuation in Part of U.S. Utility patent application Ser. No. 10/823,509 filed on Apr. 12, 2004, which is a Continuation In Part of Ser. No. 10/755,045 filed on Jan. 9, 2004, now abandoned, but which was a Continuation of Ser. No. 09/606,429 filed on Jun. 28, 2000, now U.S. Pat. No. 6,677,606.

### CONTRACTUAL ORIGIN OF THE INVENTION

The United States Government has rights in this invention pursuant to Contract No. W-31-109-ENG-38 between the University of Chicago and Argonne National Laboratory.

### BACKGROUND OF THE INVENTION

1. Field of the Invention

The present invention relates to a method for interacting biomolecules with inorganic materials, and more specifically, the present invention relates to a method for detecting changes in genetic material and manipulating the genetic material using semiconductors.

2. Background of the Invention

Exceptional electronic, optical, chemical and biological activities stem from materials scaled to nanoscaled dimensions, for example, dimensions ranging in size to less than 1000 nanometers.

The inventors have previously reported on the synthesis of semi-conductor particles having varied physical morphologies and interesting surface properties and reactivity. These reports are found in N. M. Dimitrijevic, et al., J. Am. Chem. Soc. 2005, 127, pp 1344; Z. V. Saponjic et al., Adv. Mater., 2005, 17, pp 111; T. Rajh, et al., J. Phys. Chem. B, 2002, 106, 10 543; and T. Paunesku et al., Nat. Mater., 2003, 2, 343, all incorporated herein by reference.

U.S. Pat. No. 6,667,606 B1 awarded to some of the inventors, and incorporated herein by reference, discloses nanoparticle:biomolecule composites exhibiting charge transfer characteristics at various excitation levels. Specifically, hybrid nanocomposites were developed that electronically link titanium dioxide nanoparticles to DNA oligonucleotides. TiO₂ is good for practical applications, because not only does it have photocatalytic properties, but it is also inexpensive, nontoxic, and photostable. Since TiO₂ nanoparticles are photoresponsive, they act as reporters of the electronic properties of the biomolecule.

Molecular recognition of biomolecules and their site selective bindings have unique applications in the fields of patterning, genome sequencing, and drug affinity studies. DNA oligonucleotides are especially promising because of their inherent programmability features of the nucleic-acid-based recognition system.

Single Nucleotide Polymorphisms (SNPs) are small changes in one's genetic makeup. They occur when one nucleobase replaces another in a sequence. For humans, SNPs show up in more than 1% of the population. However, most of the time, these mutations do not pose a significant health threat because they do not occur in the “coding sequences”, which compose 3%-5% of a person's DNA. However, when mutations do occur in these coding regions, protein synthesis can be altered, giving rise to higher chances for diseases such as breast cancer.

SNPs do not directly cause disease, which instead results from a combination of genetic, environmental, and lifestyle factors. However, they do indicate one's susceptibility or resistance to certain diseases and influence the severity and progression of the disease.

So far, the primary SNP detection protocols include Allele Specific Hybridization, Allele Specific Oligonucleotide Ligation, primer extension, and sequencing. However, these methods are costly, time consuming, and not the most sensitive.

A need exists in the art for a method to detect nucleotide polymorphisms which provides nearly real-time results. Also, the method should confer a sensitivity to reduce false positives and false negatives to absolute minimums.

### SUMMARY OF THE INVENTION

An object of the present invention is to provide a method for detecting nucleotide sequence anomalies that overcomes many of the disadvantages of the prior art.

Another object of the present invention is to provide a metal oxide surface between 300 nm and 400 nm long and between 40 nm and 60 nm wide for coupling with organic moieties. A feature of the surface is that it is produced without contaminants or other matter on its surface. An advantage of the invented surface is that it facilitates direct contact with the organic moieties, thereby enhancing electrical communication therebetween.

Yet another object of the present invention is to provide an electrical switch comprising inorganic metal oxide and organic compounds. A feature of the invention is that the switch is activated when exposed to radiation of a predetermined frequency. An advantage of the invention is that the switch is small enough to be used in situ and in vivo to direct electron flow to targeted tissues.

Still another object of the present invention is to provide a method for determining the location and/or number of mutations in a nucleotide. A feature of the invention is that the determination is done in a matter of a few seconds. An advantage of the invention is that the relatively longer duration experienced when using conventional analysis methods is truncated.

Another object of the present invention is to provide a method for detecting the presence of mutations which are the hallmarks of certain diseases. A feature of the invention is the ability to detect a single nitrogenous heterocyclic base anomaly by observing a decrease in conductivity of the nucleic acid strand compared to a nonmutated nucleic acid strand. An advantage of the invention is that it is tailored to detect mutation types, and not just mutation presence.

Briefly, the invention provides a method for detecting anomalies in genetic material, the method comprising supplying the genetic material; establishing electronic communication between the genetic material and a semi-conductor particle, such as a metal oxide, so as to create a composite; contacting the composite with a first metal ion; and subjecting the contacted composite to energy in an amount and for a time sufficient to reduce the first metal ion to a first elemental metal.

Also provided is a device for detecting single nucleotide mismatching in genetic material, the device comprising a semiconductor particle; a ligand attached to the particle; the genetic material in electronic communication with the ligand so as to form an organic-inorganic composite; metal ion in electronic communication with the composite; and means for energizing the semiconductor particle for a time sufficient to cause said metal ion to plate on the semiconductor particle.

### DESCRIPTION OF THE DRAWING

The present invention together with the above and other objects and advantages may best be understood from the following detailed description of the embodiment of the invention illustrated in the drawing, wherein:

FIG. 1 is a schematic diagram of electron flow in a dopamine-semiconductor construct, in accordance with features of the present invention;

FIG. 2. is a schematic diagram of electron flow in a DNA-dopamine-semiconductor construct, in accordance with features of the present invention;

FIG. 3A is a schematic depiction of metal deposition based on proximity of nucleic-acid electron donors to a semiconductor, in accordance with features of the present invention;

FIG. 3B is a graph depicting dependence of metal deposition based on charge separation distance on a nucleic acid-semiconductor construct, in accordance with features of the present invention;

FIG. 4A is a photomicrograph-schematic view depicting visual means for determining metal deposition based on number of charge hopping sites on nucleic acid molecules, in accordance with features of the present invention;

FIG. 4B is a graph showing extent of metal deposition in direct proportion to the number of charge hopping sites on nucleic acid molecules, in accordance with features of the present invention;

FIG. 4C is a graph showing the relationship of decreasing distance between adenine and thymine moieties on a nucleic acid strand and reduction of ambient metal ions to solid metal, in accordance with features of the present invention;

FIG. 5A is a photomicrograph depicting metal deposition and lack of metal deposition in the presence of guanine hopping sites in DNA;

FIG. 5B is a graph depicting metal deposition and lack of metal deposition in the presence of mismatches in DNA; and

FIG. 6 is a graph depicting the spectra fingerprints relating to the number of oligonucleotides attached to a metal oxide particle, in accordance with features of the present invention.

### DETAILED DESCRIPTION OF THE INVENTION

A method to control charge transfer reactions in DNA using semiconductors is provided. The present invention takes advantage of photocatalytic properties of semiconductor particles such as TiO₂ to detect single nucleic acid substitutions in genetic material. The method also can detect base pair anomalies such as mismatches, inasmuch as such mismatches stymie (and usually slow down) charge transfer rates. When mismatches do not exist, the intact base pair(s) facilitates charge hopping along the nucleic acid molecule, thereby increasing charge transfer rates. This enhanced charge hopping or charge transfer along a nucleic acid strand manifests as the construct's ability to reduce metal ions to elemental metal or solid metal alloy. Generally, higher plating of solid metal equates with higher conductance characteristics of the nucleic acid strand. Conversely, lower plating observations or no solid metal formation equates with the presence of mismatches which tend to confer insulative (i.e., nonconductive) characteristics to the effected strand.

The invention comprises solutions of hybrid nanocomposites (ranging in size from 2 nm to 100 nm) in electrical communication with metal ions. In one embodiment, the hybrid nanocomposites consist of mismatched oligonucleotides linked to TiO2 via dopamine. Solutions of hybrid nanocomposites were illuminated or otherwise subjected to excitation energies, all in the presence of the metal ions. By analyzing the plating or deposition of the ions as elemental metal onto the semiconductors, the inventors determine the presence of specific mismatches. Separately, the construct is capable of inducing site-specific redox chemistry in TiO₂-bound biological molecules, resulting in biological catalysis. This catalysis is the result of light-induced charge separation within the construct.

The presence of the mismatches prevents hole transfer from ligand molecules (positioned intermediate the semiconductor and the biological material) to the semiconductor, thereby extending the charge separations of the electron carriers on the hybrid construct. The nanocomposites consist of mismatched oligonucleotides linked to the semiconductor particles via dopamine. The oligonucleotides can be parts of DNA, RNA, and other material containing heterocyclic compounds such as nitrogenous bases, including, but not limited to purine and pyrimidine moieties. Dopamine serves as a conductive ligand.

In an embodiment of the present invention, a mismatch is defined as when a nucleobase is replaced with other while the change does not occur in the complementary chain of a double helix nucleic acid molecule. In this embodiment, when the change occurs in a single strand (target DNA), the strand is duplexed or otherwise probed with a known sequence (probe DNA) and if the result of the charge transfer behaves differently that expected for fully matched DNA, a mismatch is detected.

A salient feature of the device and method is the attachment of nucleic acid-based molecules to semiconductor molecules. Below is a protocol for attaching a TiO2 nanoparticle to a DNA oligonucleotide.

### DNA/Dopamine/TiO2

### Annealing Detail

As noted supra, the inventors have constructed inorganic-organic modules which enable the vectorial transport of electrons along the module and from one constituent of the module to another in a predetermined direction. The constituents are either covalently or noncovalently linked to each other. The semi-conductor particle which has various electronic ground and excited states, is combined with biological molecules or other organic material to produce the hybrid nanocomposite.

A ligand is utilized as a linker molecule between the nucleic acid moiety and the semi-conductor particle. Bidentate or tridentate modifiers, of the type discussed in U.S. Pat. No. 6,677,606, and incorporated herein in its entirety, are suitable. For the sake of illustration only, hydroxyl phenyls such as dopamine are utilized as a nucleophillic (i.e. electron donating) linker moiety. The selection of dopamine, the use of specific oligonucleotide structures, reagents and reagent concentrations are recounted here to allow the reader to understand the invented method, repeat the invented method and reproduce the hybrid nanocomposite detector. A myriad of substitutions of the individual parts of the construct are suitable as can be perceived by persons skilled in the chemical arts. For example, suitable bidentate molecules include, but are not limited to, enediol ligands such as dopamine, dihydroxyphenyl acetic acid (DOPAC), and combinations thereof. Other bidentate candidates are selected from the polyols, including, but not limited to, glycerol, polyethylene glycol, and glucose.

### Metal Deposition Detail

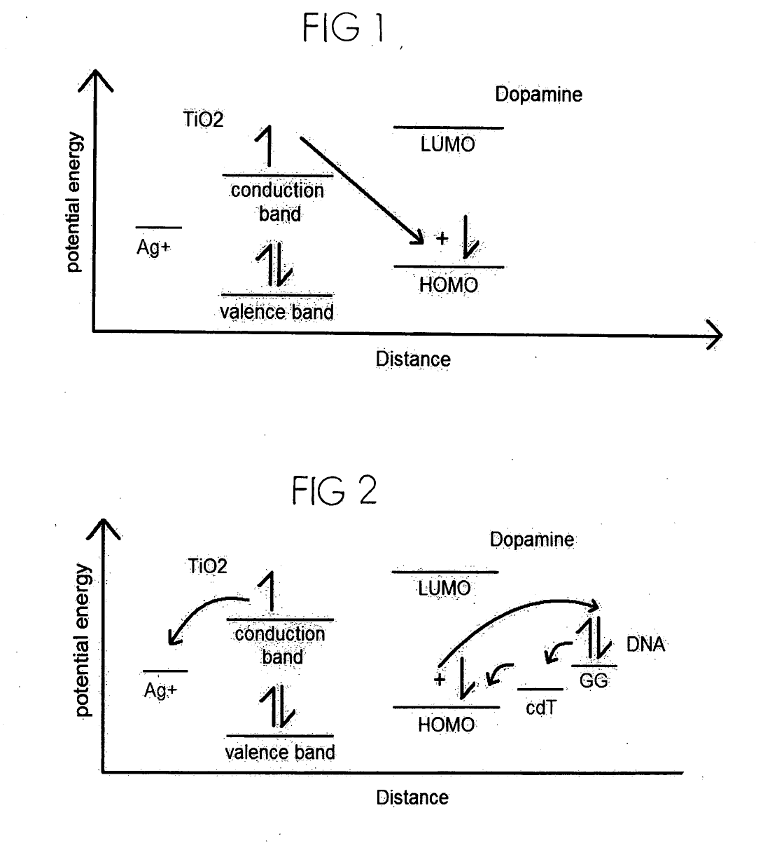

FIGS. 1 and 2 provide schematic representations of the invented method. FIG. 1 represents the energy state of the invented hybrid nanocomposite not in the presence of nucleic acid. FIG. 1 shows that absorption of light of a sufficient energy (1.6 eV when TiO₂ modified with dopamine is used) results in the formation of electron/hole pairs. When this energy illuminates, irradiates, or otherwise interacts with TiO₂, an electron from the highest occupied molecular orbital (HOMO) of dopamine is promoted to the conduction band (highest unoccupied shell) of the metal. Even with metal ions present in solution, no metal deposition on the semiconductor will occur because the attraction between the positively charged hole in the dopamine and the excited electron in the conduction band of the metal is stronger than that electron's attraction to metal ion.

However, and as depicted in FIG. 2, when a hybrid nanocomposite is made comprising TiO₂, dopamine and DNA with a GG base sequence along the same strand of a nucleic acid molecule, metal deposition becomes possible. (While FIG. 2 depicts silver as the metal ion reduced, this is for illustrative purposes only. Other metal candidates include, but are not limited to mercury, copper, gold, alloys thereof and combinations of these metals, including silver.)

Due to the potential difference and short distance between GG and dopamine through covalent bonding, GG (which has a higher potential energy than dopamine) donates electrons to dopamine. As a result, the positive hole which used to be in the HOMO of dopamine moves to GG, subsequently increasing the distance between the positively charged hole and the excited electron in the conduction band of TiO₂. (In other words, the absorption of light by inventor-fabricated nanocrystallites results in charge separation, with holes (positive charge) being localized on the distally positioned oligonucleotide.) While the inventors chose guanine-containing sites on genetic material to facilitate site selective photooxidation of DNA (due to the relative ease of oxidation of guanine compared to other nucleobases) other nucleobases are also suitable electron donors.

One embodiment of the invention is particularly suited to detect a G mismatch, whereby the mismatch results in an absence of guanine and whereby the mismatch is positioned intermediate a distally positioned GG sequence and the ligand molecule. In this embodiment, a hole can not “jump” over the mismatch region to reach the final destination, i.e., the most distal GG hole sink. The inventors have determined that the intermediate G mismatch region imparts an insulative region to the strand, thereby rendering the chain less conductive such that the hole does not reach the sink or final GG trap.

In one embodiment, decreases of strand conductivity occur when bases are replaced with thymine (T), the most insulating base. Alternatively, an increase of conductivity occurs when T is replaced by cytosine (C). Surprisingly and unexpectedly, the inventors found that the chain is more conductive through a kink formed by mismatch base pairs.

Ultimately, the separation distance increases to a nonconductive distance such that the electron pair can no longer recombine. As a result, the attraction between the promoted electron and silver ions is greater. Furthermore, due to the potential difference between the conduction band of TiO₂ and Ag+, TiO₂ will donate electrons to the silver ions, reducing them to silver atoms which are dark in color. This is how silver deposition can be detected.

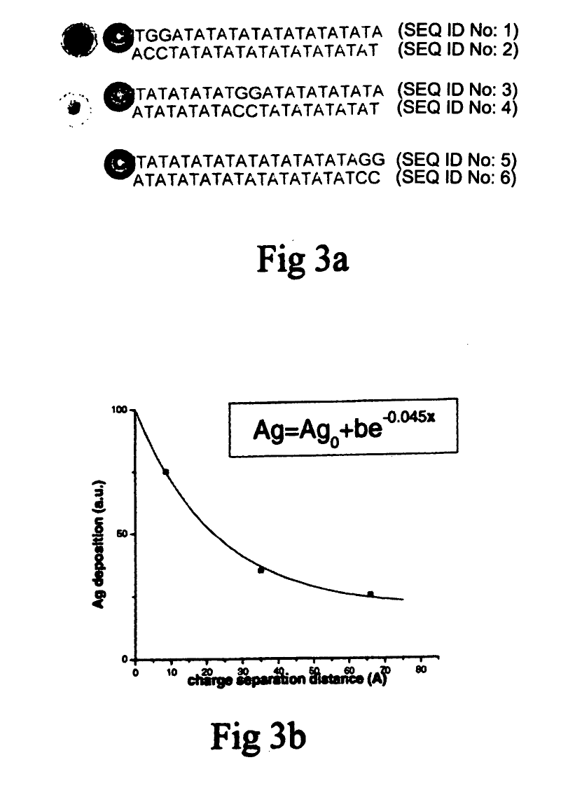

As GG moves farther away from the dopamine ligand on the DNA oligonucleotide, it becomes harder for the base pair sequence to donate an electron to the HOMO of dopamine. As is depicted in FIG. 3A, with increasing GG distance away from dopamine, and therefore from the semiconductor surface the positively charged hole remains closer to the conduction band of TiO₂. This increasing distance results in increasing the probability of charge recombination and reducing the amount of silver deposition.

FIG. 3B shows the decreasing levels of silver deposition directly proportional to the distance of the electron donating GG sequence pair from the dopamine/semi-conductor particle construct. Furthermore, in an embodiment of the invented construct, the hole hopping from dopamine to the most distal GG site is enhanced with the presence of repeating adenine units compared to when the adenine units are separated by thymine.

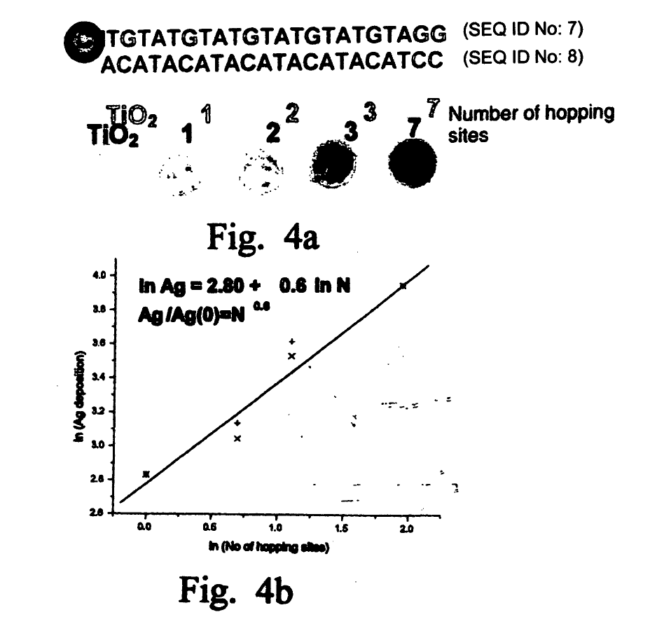

FIG. 4a shows that as G-C hopping sites are introduced in poly AT chain before a GG final hole trapping site, hole hopping becomes more efficient as observed with increased silver deposition. FIG. 4A depicts a visual increase in metal deposition. The inventors have found that the base packing along a nucleic acid strand is more compact with the increased presence of adenine, with a base-to-base distance of 3.3 Angstroms compared to a typical 3.5 Angstrom average distance. Thus, the increased presence of adenine serves as a means for decreasing the distance between base pairs and a means for enhancing strand conductivity.

FIG. 4B depicts a graphic natural logarithmic relationship between the number of charge transfer or charge hopping sites on a nucleic acid strand and the extent of metal deposition. “N” denotes the number of hopping sites.

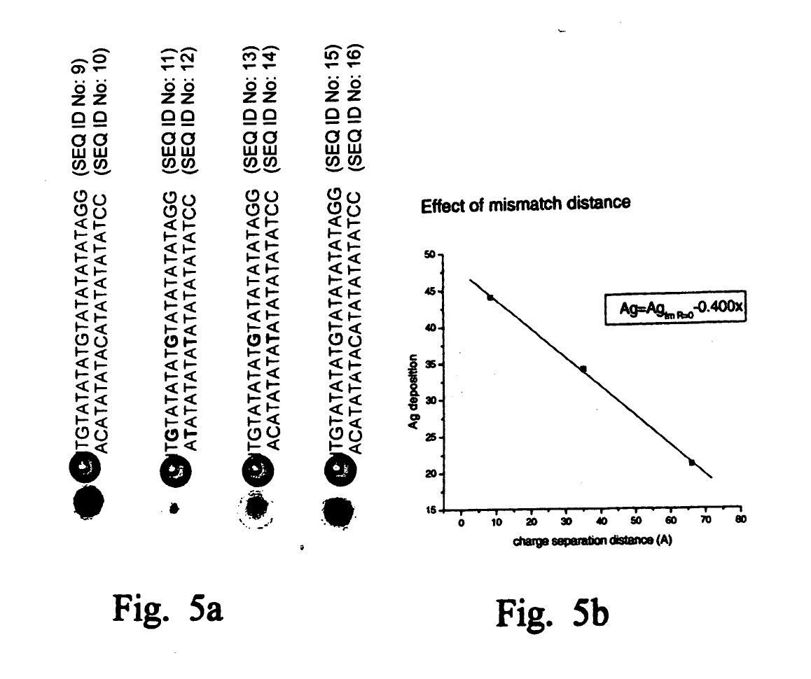

FIG. 4C is a graph depicting the extent of metal deposition in the invented construct compared to the length in angstroms of several joined adenine-thymine couplets on a strand. The ordinate of the graph depicts percentage of excitation photons which are utilized in the plating or deposition process. As such, Φ is the quantum yield of silver deposition defined as number of photons that facilitate metal ion (silver in the figure) reduction. FIG. 5A depicts the separation of G-containing hopping sites by these several A-T couplets. FIG. 4C shows that the longer the length of the A-T strands interposed between the hopping sites, the less metal deposition occurs.

Introducing G-C hopping sites makes hole hopping more efficient and as a G hopping site is introduced every 15 Å, it becomes as efficient (42%) as in the case that a final GG accepting site is placed 7 Å from the nanoparticle surface.

When one of the G-C hopping sites is replaced with the G-T mismatch, silver deposition decreases (FIG. 5A) allowing for sensitive detection of single C to T DNA sequence variation (Single Nucleotide Polymorphism, SNP).

BRCA1 and BRCA2 are human breast cancer mutations in chromosome 17 and 13, respectively. Each of them is characterized by 12 SNPs several of them C/T SNP. These SNPs can be detected using metal deposition.

### Example

Generally, genetic material adapted to receive a linker molecule with the aforementioned characteristics is utilized. For example, carboxyl-dT terminated oligonucleotides are suitable. In the laboratory, the inventors supplied the oligonucleotides in a stock solution having a concentration of about 20 μM.

Before illumination, the amino group of dopamine was linked to 4 different sets of DNA (16-20 nucleotides long) having carboxyl groups at the 5′ end (via the intermediate N-hydroxy-succinimide ester). About 50 μl of oligo stock solution, 450 μl of DMF, 5 mg of TSU (O-(N-succinimidyl)-N,N,N′,N′tetramehtylamonium tetrafluoroorate) and 7 μl of i-PrEtN (N,N′-diisopropyl amine) were combined. The solution was agitated for 6 hours at 4 degrees Celsius. Dialyze 500 μl against 50 ml of 1:1 H2O/DMF three times. Add 200 μl of dioxane, (bubble the solution with nitrogen (use small needle, check the nitrogen flow to go bubble by bubble before you start bubbling DNA solution, or) leave vials open in nitrogen atmosphere for a night). Add 7 μl of i-PrEEtN and dopamine to make 100 μM solution under nitrogen. The solution is incubated 24 hours in the dark and under nitrogen or similar fluid devoid of oxygen. The mixture is then dialyzed four times in room temperature against water under nitrogen (the water should be bubbled with nitrogen before dialysis), and then dried under vacuum. This yields oligonucleotides end-labeled with dopamine. Add 100 μl of TiO2/glycine isopropyl ether in 10 mM buffer.

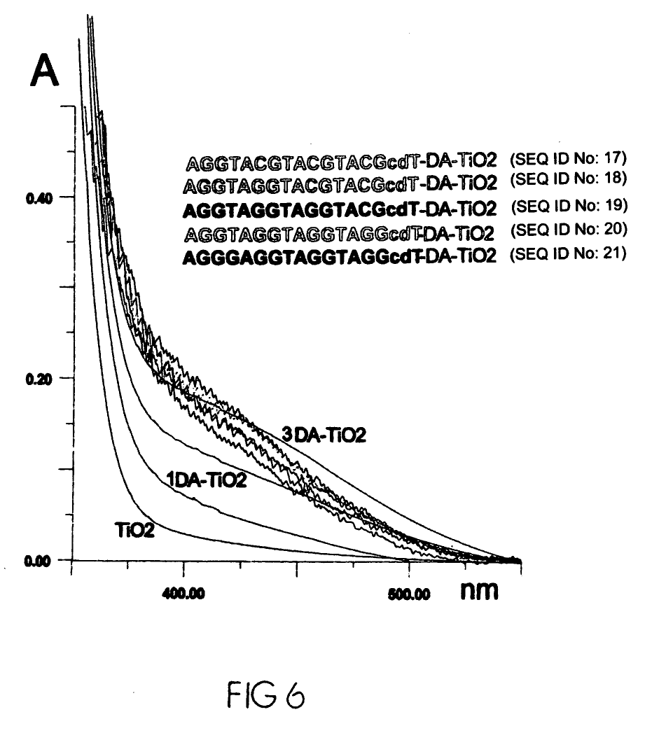

Then, DA-DNA was attached to TiO_{2.} The absorption spectroscopy shown in FIG. 6 provides a means to determine the number of oligonucleotides that are bound to TiO_{2.} Dopamine serves as a conduit of charge for bases on the DNA, and thus, charge separation is possible for the hybrid nanocomposite. In the spectroscopy, the peak of TiO₂ shifts, and this change is proportional to the number of dopamine particles that are attached to TiO₂, which indicates how many nucleic acid strands are linked to the dopamine. Though the spectroscopy indicates that the amount of DNA-DA bound to TiO₂ fluctuates (which depends on the accuracy and precision of lab techniques), the spectroscopy shows that binding was consistent, at 2-3 oligonucleotides per semi-conductor particle.

### Particle Detail

Inorganic nanoparticles shaped as spheres, rods, disks, cylinders, pyramids, cubes, multi-apex (i.e., star-like), and other predetermined shapes are suitable semiconductor substrate configurations. Methods for preparing these various shapes are disclosed in U.S. patent application Ser. No. 11/438,180 filed on May 22, 2006, the entirety of which is incorporated herein by reference. Generally the particles range in size from between 2 nanometers and 100 nanometers.

A myriad of metal oxides are suitable nanoparticle candidates, including, but not limited to TiO₂, WO₃, Fe₂O₃, ZrO₂, SnO₂, VO₂ and combinations thereof.

Surprisingly and unexpectedly, the inventors found site-specific defects located at the tips of the synthesized rods. Hereinafter referred to as “corner defects”, these anomalies are related to the size and shape of features on the particle.

The site-specific defects include a deviation from the hexa-coordinated (Octahedral) configuration of the metal atoms in the lattice such that a constraining of the atomic arrangement of the atoms occurs. This confinement occurs within less than 10 atomic layers from the tip of the synthesized particle, resulting in an under-coordinated (i.e., less than the normal Oxygen-atom contingent) atomic character to the TI metal sites. This under-coordinate causes a lengthening of the Ti—Ti distances along the longitudinal axis of the crystal.

The incompletely coordinated Ti defect sites exhibit a high affinity for oxygen-containing ligands and present the opportunity for chemical attachment and modification. For example, and as more fully disclosed in Saponjic et al., Adv. Mater. 2005, No. 8, pp 965-971 and incorporated herein by reference, oxygen-rich enediol ligands form strongly coupled conjugated structures by repairing the coordination surface via chelation. As a consequence, the intrinsic properties of the semiconductor change and new, hybrid molecular orbitals are generated by mixing the orbitals of chelating ligands and the continuum states of the metal oxides. This results in the red-shift of the absorption compared to unmodified nanocrystallites.

The electronic “topography” of these conjugated structures can provide a means for directing attachment of the ligands (such as dopamine) to certain portions of the semi-conductor particle. Specifically, the inventors found that the under-coordinated defect sites facilitate direct chemical functionalization and specifically, the Ti—Ti atom positioning in the defect site represents an optimal docking site for the enediol groups of dopamine. As such, the surface tip defect promotes the binding of dopamine exclusively to the tips of the synthesized titanium particle. Two benefits are realized as a result: First, semiconductor particles can be manipulated and connected tip-to-tip to form “chainlike” structures, as noted in the “XXX patent application, referenced supra. The formation of such chainlike structures is found in Dimitrijevic et al., J. Am. Chem. Soc. 2005, 127 pp 1344-1345, heretofore incorporated herein by reference. Thus, plating of semiconductors with a first metal can be confined to one end of a chain, while additional metals can be used in plating other regions of the chain structure.

Alternatively, tailoring dopamine attachment to a first portion of a single semiconductor particle can result in plating of the semiconductor with a first metal (supplied via a first solution of first metal ion) being confined to that first portion of the semiconductor. Then, a second metal plating operation can be conducted, whereby the partially-plated construct is exposed to a second metal ion solution. Upon exposure to a second round of radiation, unplated regions of the partially plated semiconductor is plated with the second metal.

This plurality of different metal plating provides a means for optimizing the detection capabilities of the invented hybrid nanocomposite.

In one embodiment, a TiO₂ particle is provided, having the corner defects discussed supra. The corner defects facilitate covalent bonding with dopamine via a bidentate complex of dopamine OH groups with the under-coordinated TI surface atoms. Upon bonding with dopamine (one titanium atom to two hydroxyl groups on the dopamine), the constrained configuration of the Titanium atoms involved relax to the original octahedral lattice configuration, resulting in the formation of a very stable ligand-to-metal complex, estimated at 25 kcal/mole. This relaxation serves as a means for eliminating surface trapping centers which would otherwise constrain mobile electrons.

This dopamine preparation of the tips of the Titanium particle facilitates covalent bonding of genetic material such as nucleic acid, DNA, etc., to titanium particle via an intermediately positioned dopamine moiety, via a condensation reaction. Alternatively, dopamine can first be bound to the genetic material form a dopamine-DNA construct, with that construct then bound to the constrained sites of titanium.

While the invention has been described with reference to details of the illustrated embodiment, these details are not intended to limit the scope of the invention as defined in the appended claims.

1. A method for detecting anomalies in genetic material, the method comprising:
a) supplying the genetic material;
b) establishing electronic communication between the genetic material and a semi-conductor particle so as to create a composite;
c) contacting the composite with a first metal ion; and
d) subjecting the contacted composite to energy in an amount and for a time sufficient to reduce the first metal ion to a first elemental metal.

2. The method as recited in claim 1 wherein a ligand is positioned intermediate the genetic material and the semiconductor.

3. The method as recited in claim 1 wherein the anomalies consist of a single nucleotide mismatch.

4. The method as recited in claim 1 wherein the genetic material is a compound selected from the group consisting of DNA, RNA, nucleic acid, peptides, and combinations thereof.

5. The method as recited in claim 1 wherein the metal is silver, mercury or copper or gold or combinations thereof.

6. The method as recited in claim 1 wherein the inorganic particles are oxides selected from the group consisting of TiO₂, WO₃, Fe₂O₃, ZrO₂, SnO₂, VO₂, and combination thereof.

7. The method as recited in claim 1 wherein the first elemental metal deposits on a first region of the semiconductor to form a first plated semiconductor.

8. The method as recited in claim 8 further comprising:
a) contacting a second metal ion to the first plated semiconductor to form a second contacted composite;
b) subjecting the contacted composite to energy in an amount and for a time sufficient to reduce the second metal ion to a second elemental metal.

9. A device for detecting single nucleotide mismatching in genetic material, the device comprising:
a) a semiconductor particle;
b) a ligand attached to the particle;
c) the genetic material in electronic communication with the ligand so as to form an organic-inorganic composite;
d) metal ion in electronic communication with the composite; and
e) means for energizing the semiconductor particle for a time sufficient to cause said metal ion to plate on the semiconductor particle.

10. The device as recited in claim 9 wherein the semiconductor particle is an oxide selected from the group consisting of TiO₂, WO₃, Fe₂O₃, ZrO₂, SnO₂, VO₂, and combinations thereof.

11. The device as recited in claim 9 wherein the ligand is a bidentate molecule selected from the group consisting of enediol ligands such as dopamine, DOPAC, and combinations thereof.

12. The structure as recited in claim 9 wherein the metal ion is silver, or mercury, or copper, or gold, or combinations thereof.


SEQUENCE LISTING (21 sequences)

SEQ ID NO: 1 - DNA, 22 bp, Artificial Sequence
  Sample DNA Seach String
tggatatatatatatatatata

SEQ ID NO: 2 - DNA, 22 bp, Artificial Sequence
  Sample DNA Seach String
tatatatatatatatatatcca

SEQ ID NO: 3 - DNA, 22 bp, Artificial Sequence
  Sample DNA Seach String
tatatatatggatatatatata

SEQ ID NO: 4 - DNA, 22 bp, Artificial Sequence
  Sample DNA Seach String
tatatatatatccatatatata

SEQ ID NO: 5 - DNA, 22 bp, Artificial Sequence
  Sample DNA Seach String
tatatatatatatatatatagg

SEQ ID NO: 6 - DNA, 22 bp, Artificial Sequence
  Sample DNA Seach String
cctatatatatatatatatata

SEQ ID NO: 7 - DNA, 22 bp, Artificial Sequence
  Sample DNA Seach String
tgtatgtatgtatgtatgtagg

SEQ ID NO: 8 - DNA, 22 bp, Artificial Sequence
  Sample DNA Seach String
cctacatacatacatacataca

SEQ ID NO: 9 - DNA, 22 bp, Artificial Sequence
  Sample DNA Seach String
tgtatatatgtatatatatagg

SEQ ID NO: 10 - DNA, 22 bp, Artificial Sequence
  Sample DNA Seach String
cctatatatatacatatataca

SEQ ID NO: 11 - DNA, 22 bp, Artificial Sequence
  Sample DNA Seach String
tgtatatatgtatatatatagg

SEQ ID NO: 12 - DNA, 22 bp, Artificial Sequence
  Sample DNA Seach String
cctatatatatatatatatata

SEQ ID NO: 13 - DNA, 22 bp, Artificial Sequence
  Sample DNA Seach String
tgtatatatgtatatatatagg

SEQ ID NO: 14 - DNA, 22 bp, Artificial Sequence
  Sample DNA Seach String
cctatatatatatatatataca

SEQ ID NO: 15 - DNA, 22 bp, Artificial Sequence
  Sample DNA Seach String
tgtatatatgtatatatatagg

SEQ ID NO: 16 - DNA, 22 bp, Artificial Sequence
  Sample DNA Seach String
cctatatatatacatatataca

SEQ ID NO: 17 - DNA, 16 bp, Artificial Sequence
  Sample DNA Seach String
tgcatgcatgcatgga

SEQ ID NO: 18 - DNA, 16 bp, Artificial Sequence
  Sample DNA Seach String
tgcatgcatggatgga

SEQ ID NO: 19 - DNA, 16 bp, Artificial Sequence
  Sample DNA Seach String
tgcatggatggatgga

SEQ ID NO: 20 - DNA, 16 bp, Artificial Sequence
  Sample DNA Seach String
tggatggatggatgga

SEQ ID NO: 21 - DNA, 16 bp, Artificial Sequence
  Sample DNA Seach String
tggatggatggaggga